(19) United States
(12) Patent Application Publication
(10) Pub. No.: US 2024/0087669 A1
(43) Pub. Date: Mar. 14, 2024

(54) MULTI-MICRO-ORGAN CULTURE AND INTERACTION BIG DATA ANALYSIS SYSTEM BASED ON CLOUD NATIVE

(71) Applicants: Yulin CAO, Jiyuan (CN); Tangyi Holding (Shenzhen) Co., Ltd., Shenzhen (CN)

(72) Inventor: Yulin CAO, Beijing (CN)

(73) Assignees: Yulin CAO, Jiyuan (CN); Tangyi Holding (Shenzhen) Co., Ltd., Shenzhen (CN)

(21) Appl. No.: 18/093,829

(22) Filed: Jan. 6, 2023

(30) Foreign Application Priority Data
Sep. 14, 2022 (CN) 202211119908.5

(51) Int. Cl. G16B 5/00; G16B 40/20

(52) U.S. Cl. CPC G16B 5/00; G16B 40/20; G16H 10/40

(57) ABSTRACT

A multi-micro-organ culture and interaction big data analysis system based on cloud native includes a multi-micro-organ culture database, a learning model establishment module, a feature extraction terminal, a multi-micro-organ visualization terminal and a multi-micro-organ learning terminal, the multi-micro-organ culture database is connected to the learning model establishment module by means of a signal line, the learning model establishment module is connected to the feature extraction terminal by means of a signal line, the feature extraction terminal is connected to the multi-micro-organ visualization terminal and the multi-micro-organ learning terminal separately by means of signal lines, and the feature extraction terminal includes a micro-organ function extraction module, a micro-organ interaction extraction module and a medicine response extraction module. According to the system, a multi-agent reinforcement learning algorithm can be researched and established, micro-organ multi-modal data can be integrated and analyzed, and interaction system state and feature information can be extracted.

### CROSS REFERENCE TO THE RELATED APPLICATIONS

This application is based upon and claims priority to Chinese Patent Application No. 202211119908.5, filed on Sep. 14, 2022, the entire contents of which are incorporated herein by reference.

### TECHNICAL FIELD

The disclosure relates to the technical field of big data analysis, and particularly to a multi-micro-organ culture and interaction big data analysis system based on cloud native.

### BACKGROUND

As one form of generalized tissue culture, micro-organ culture means isolated culture of part of a living body. Specifically, part or whole micro-organ is cultured under the condition of not damaging normal tissue structures, that is, a three-dimensional structure of tissue is still maintained, and micro-organ functions in various states are simulated.

An analysis system is required in multi-micro-organ culture and interaction big data analysis. However, the existing analysis system cannot establish a multi-agent reinforcement learning algorithm, cannot integrate and analyze micro-organ multi-modal data, and cannot extract interaction system state and feature information. Moreover, the interaction system state and feature information cannot be converted into functional quantitative indexes for evaluating application such as micro-organ interaction research and medicine screening, causing the poor analysis effect. Therefore, the existing analysis system has space for improvement.

### SUMMARY

The objective of the disclosure is to provide a multi-micro-organ culture and interaction big data analysis system based on cloud native, so as to solve the defects in the prior art. The disclosure has the advantages that a multi-agent reinforcement learning algorithm may be researched and established, micro-organ multi-modal data may be integrated and analyzed, interaction system state and feature information may be extracted, and the interaction system state and feature information may be converted into functional quantitative indexes for evaluating application such as micro-organ interaction research and medicine screening, so as to improve comprehensiveness of data analysis.

In order to realize the above objective, the disclosure employs the technical solutions as follows:

a multi-micro-organ culture and interaction big data analysis system based on cloud native includes a multi-micro-organ culture database, a learning model establishment module, a feature extraction terminal, a multi-micro-organ visualization terminal and a multi-micro-organ learning terminal, the multi-micro-organ culture database is connected to the learning model establishment module by means of a signal line, the learning model establishment module is connected to the feature extraction terminal by means of a signal line, the feature extraction terminal is connected to the multi-micro-organ visualization terminal and the multi-micro-organ learning terminal separately by means of signal lines, and the feature extraction terminal includes a micro-organ function extraction module, a micro-organ interaction extraction module and a medicine response extraction module.

The disclosure is further configured as follows: the multi-micro-organ culture database includes a micro-organ data receiving unit and a micro-organ data storage unit, the micro-organ data receiving unit is used for receiving processed data, such as parameter information of a micro-organ specificity environment, amplification information of micro-organs, material exchange information between the micro-organs and a blood circulation system, and signal exchange information between the micro-organs and a nervous system, and the micro-organ data storage unit is used for classifying and storing received data.

The disclosure is further configured as follows: the learning model establishment module includes a reinforcement learning algorithm unit and a micro-organ data analysis unit, the reinforcement learning algorithm unit is used for establishing a multi-agent reinforcement learning algorithm, and the micro-organ data analysis unit is used for integrating and analyzing micro-organ multi-modal data.

The disclosure is further configured as follows: the micro-organ function extraction module includes a micro-organ function identification unit and a micro-organ function extraction unit, the micro-organ function identification unit is used for identifying the parameter information of the micro-organ specificity environment, the amplification information of the micro-organs, the material exchange information between the micro-organs and the blood circulation system, and the signal exchange information between the micro-organs and the nervous system, and the micro-organ function extraction unit is used for extracting identified micro-organ function data.

The disclosure is further configured as follows: the micro-organ interaction extraction module includes a micro-organ interaction identification unit and a micro-organ interaction extraction unit, the micro-organ interaction identification unit is used for identifying micro-organ interaction data, and the micro-organ interaction extraction unit is used for extracting identified micro-organ interaction data.

The disclosure is further configured as follows: the medicine response extraction module includes a medicine response identification unit and a medicine response extraction unit, the medicine response identification unit is used for identifying medicine response data, and the medicine response extraction unit is used for extracting identified medicine response data.

The disclosure is further configured as follows: the multi-micro-organ visualization terminal includes a micro-organ data decoding unit, a micro-organ data display unit and a data display optimization unit, the micro-organ data decoding unit is used for decoding and converting analyzed data into a format that may be displayed, and the micro-organ data display unit is used for displaying decoded data.

The disclosure is further configured as follows: the multi-micro-organ learning terminal includes a multi-micro-organ learning unit, and the multi-micro-organ learning unit is used for finding an optimal solution of a control parameter through machine learning and big data analysis.

A working method of the multi-micro-organ culture and interaction big data analysis system based on cloud native includes the following steps:
step 1, store processed data by a multi-micro-organ culture database, and call related data from the database after storage;
step 2, research and establish a multi-agent reinforcement learning algorithm, integrate and analyze micro-organ multi-modal data, extract interaction system state and feature information, and convert the interaction system state and feature information into functional quantitative indexes for evaluating application such as micro-organ interaction research and medicine screening; and
step 3: display functional quantitative index data after conversion by a multi-micro-organ visualization terminal, carry out autonomous learning by the system according to related data after display, and then carry out autonomous optimization, so as to complete multi-micro-organ culture and interaction big data analysis.

The disclosure has the beneficial effects: the disclosure discloses a multi-micro-organ culture and interaction big data analysis system based on cloud native, a multi-agent reinforcement learning algorithm may be researched and established, micro-organ multi-modal data may be integrated and analyzed, interaction system state and feature information may be extracted, and the interaction system state and feature information may be converted into functional quantitative indexes for evaluating application such as micro-organ interaction research and medicine screening, so as to improve comprehensiveness of data analysis.

### BRIEF DESCRIPTION OF THE DRAWINGS

FIG. 1 is a schematic diagram of an overall structure of a multi-micro-organ culture and interaction big data analysis system based on cloud native provided in the disclosure;

FIG. 2 is a schematic structural diagram of a learning model establishment module of a multi-micro-organ culture and interaction big data analysis system based on cloud native provided in the disclosure;

FIG. 3 is a schematic structural diagram of a multi-micro-organ culture database of a multi-micro-organ culture and interaction big data analysis system based on cloud native provided in the disclosure; and

FIG. 4 is a schematic structural diagram of a multi-micro-organ visualization terminal of a multi-micro-organ culture and interaction big data analysis system based on cloud native provided in the disclosure.

### DETAILED DESCRIPTION OF THE EMBODIMENTS

The technical solution of the patent will be further described in detail below in combination with the specific embodiments.

Examples of the patent will be described in detail below, and instances of the examples are shown in accompanying drawings, throughout which identical or similar reference numerals denote identical or similar elements or elements having identical or similar functions. The examples described below with reference to the accompanying drawings are exemplary and are merely used to explain the patent, but cannot be construed as limiting the patent.

In the description of the patent, it is to be understood that orientation or positional relations indicated by the terms “center”, “upper”, “lower”, “front”, “rear”, “left”, “right”, “vertical”, “horizontal”, “top”, “bottom”, “inside”, “outside”, etc. are based on orientation or positional relations shown in the accompanying drawings, are merely for facilitating the description of the patent and simplifying the description, rather than indicating or implying that an apparatus or element referred to must have a particular orientation or be constructed and operated in a particular orientation, and therefore will not be construed as limiting the patent.

In the description of the patent, it should be noted that unless explicitly specified and defined otherwise, the terms “mount”, “connected”, “connect”, and “arranged” should be understood broadly, for example, they can mean fixed connection and arrangement, detachable connection and arrangement, or integral connection and arrangement. For those of ordinary skill in the art, the specific meanings of the above terms in the patent could be understood according to specific circumstances.

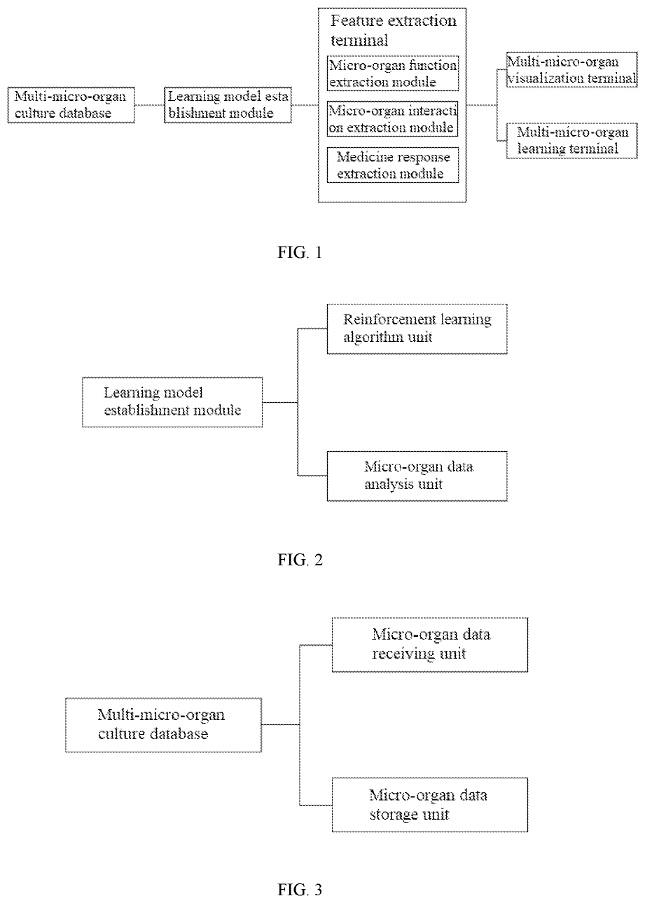

With reference to FIG. 1. a multi-micro-organ culture and interaction big data analysis system based on cloud native includes a multi-micro-organ culture database, a learning model establishment module, a feature extraction terminal, a multi-micro-organ visualization terminal and a multi-micro-organ learning terminal, the multi-micro-organ culture database is connected to the learning model establishment module by means of a signal line, the learning model establishment module is connected to the feature extraction terminal by means of a signal line, the feature extraction terminal is connected to the multi-micro-organ visualization terminal and the multi-micro-organ learning terminal separately by means of signal lines, and the feature extraction terminal includes a micro-organ function extraction module, a micro-organ interaction extraction module and a medicine response extraction module.

With reference to FIG. 2, the learning model establishment module includes a reinforcement learning algorithm unit and a micro-organ data analysis unit, the reinforcement learning algorithm unit is used for establishing a multi-agent reinforcement learning algorithm, and the micro-organ data analysis unit is used for integrating and analyzing micro-organ multi-modal data. The micro-organ function extraction module includes a micro-organ function identification unit and a micro-organ function extraction unit, the micro-organ function identification unit is used for identifying parameter information of a micro-organ specificity environment, amplification information of micro-organs, material exchange information between the micro-organs and a blood circulation system, and signal exchange information between the micro-organs and a nervous system, and the micro-organ function extraction unit is used for extracting identified micro-organ function data. The micro-organ interaction extraction module includes a micro-organ interaction identification unit and a micro-organ interaction extraction unit, the micro-organ interaction identification unit is used for identifying micro-organ interaction data, and the micro-organ interaction extraction unit is used for extracting identified micro-organ interaction data.

With reference to FIG. 3, the multi-micro-organ culture database includes a micro-organ data receiving unit and a micro-organ data storage unit, the micro-organ data receiving unit is used for receiving processed data, such as the parameter information of the micro-organ specificity environment, the amplification information of the micro-organs, the material exchange information between the micro-organs and the blood circulation system, and the signal exchange information between the micro-organs and the nervous system, and the micro-organ data storage unit is used for classifying and storing received data.

In the example, the medicine response extraction module includes a medicine response identification unit and a medicine response extraction unit, the medicine response identification unit is used for identifying medicine response data, and the medicine response extraction unit is used for extracting identified medicine response data.

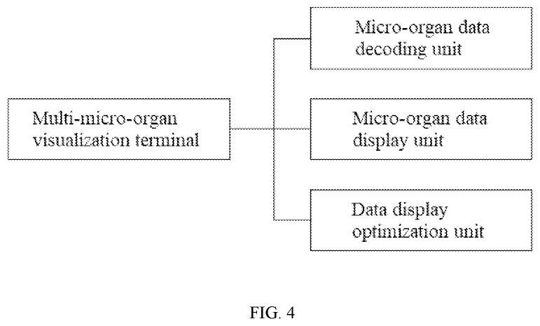

With reference to FIG. 4, the multi-micro-organ visualization terminal includes a micro-organ data decoding unit, a micro-organ data display unit and a data display optimization unit, the micro-organ data decoding unit is used for decoding and converting analyzed data into a format that may be displayed, and the micro-organ data display unit is used for displaying decoded data. The multi-micro-organ learning terminal includes a multi-micro-organ learning unit, and the multi-micro-organ learning unit is used for finding an optimal solution of a control parameter through machine learning and big data analysis.

A working method of the multi-micro-organ culture and interaction big data analysis system based on cloud native includes the following steps:
step 1, store processed data by a multi-micro-organ culture database, and call related data from the database after storage;
step 2, research and establish a multi-agent reinforcement learning algorithm, integrate and analyze micro-organ multi-modal data, extract interaction system state and feature information, and convert the interaction system state and feature information into functional quantitative indexes for evaluating application such as micro-organ interaction research and medicine screening; and
step 3: display functional quantitative index data after conversion by a multi-micro-organ visualization terminal, carry out autonomous learning by the system according to the related data after display, and then carry out autonomous optimization, so as to complete multi-micro-organ culture and interaction big data analysis.

What is mentioned above is merely optimal specific embodiments of the disclosure, but is not intended to limit the scope of protection of the disclosure. Any equivalent substitutions or changes made by those skilled in the art according to the technical solution of the disclosure and the inventive concept thereof within the scope of technology disclosed in the disclosure should fall within the scope of protection of the disclosure.

What is claimed is:

1. A multi-micro-organ culture and interaction big data analysis system based on cloud native, comprising a multi-micro-organ culture database, a learning model establishment module, a feature extraction terminal, a multi-micro-organ visualization terminal and a multi-micro-organ learning terminal, wherein the multi-micro-organ culture database is connected to the learning model establishment module by a first signal line, the learning model establishment module is connected to the feature extraction terminal by a second signal line, the feature extraction terminal is connected to the multi-micro-organ visualization terminal and the multi-micro-organ learning terminal separately by third signal lines, and the feature extraction terminal comprises a micro-organ function extraction module, a micro-organ interaction extraction module and a medicine response extraction module.

2. The multi-micro-organ culture and interaction big data analysis system based on cloud native according to claim 1, wherein the multi-micro-organ culture database comprises a micro-organ data receiving unit and a micro-organ data storage unit, wherein
the micro-organ data receiving unit is configured for receiving processed data, wherein the processed data comprise parameter information of a micro-organ specificity environment, amplification information of micro-organs, material exchange information between the micro-organs and a blood circulation system, and signal exchange information between the micro-organs and a nervous system; and
the micro-organ data storage unit is configured for classifying and storing received data.

3. The multi-micro-organ culture and interaction big data analysis system based on cloud native according to claim 2, wherein the learning model establishment module comprises a reinforcement learning algorithm unit and a micro-organ data analysis unit, wherein the reinforcement learning algorithm unit is configured for establishing a multi-agent reinforcement learning algorithm, and the micro-organ data analysis unit is configured for integrating and analyzing micro-organ multi-modal data.

4. The multi-micro-organ culture and interaction big data analysis system based on cloud native according to claim 3, wherein the micro-organ function extraction module comprises a micro-organ function identification unit and a micro-organ function extraction unit, wherein
the micro-organ function identification unit is configured for identifying the parameter information of the micro-organ specificity environment, the amplification information of the micro-organs, the material exchange information between the micro-organs and the blood circulation system, and the signal exchange information between the micro-organs and the nervous system; and
the micro-organ function extraction unit is configured for extracting identified micro-organ function data.

5. The multi-micro-organ culture and interaction big data analysis system based on cloud native according to claim 4, wherein the micro-organ interaction extraction module comprises a micro-organ interaction identification unit and a micro-organ interaction extraction unit, wherein the micro-organ interaction identification unit is configured for identifying micro-organ interaction data, and the micro-organ interaction extraction unit is configured for extracting identified micro-organ interaction data.

6. The multi-micro-organ culture and interaction big data analysis system based on cloud native according to claim 5, wherein the medicine response extraction module comprises a medicine response identification unit and a medicine response extraction unit, wherein the medicine response identification unit is configured for identifying medicine response data, and the medicine response extraction unit is configured for extracting identified medicine response data.

7. The multi-micro-organ culture and interaction big data analysis system based on cloud native according to claim 6, wherein the multi-micro-organ visualization terminal comprises a micro-organ data decoding unit, a micro-organ data display unit and a data display optimization unit, wherein
the micro-organ data decoding unit is configured for decoding and converting analyzed data into a format, wherein the format is allowed to be displayed; and
the micro-organ data display unit is configured for displaying decoded data.

8. The multi-micro-organ culture and interaction big data analysis system based on cloud native according to claim 1, wherein the multi-micro-organ learning terminal comprises a multi-micro-organ learning unit, wherein the multi-micro-organ learning unit is configured for finding an optimal solution of a control parameter through machine learning and big data analysis.

9. A working method of the multi-micro-organ culture and interaction big data analysis system based on cloud native according to claim 1, comprising the following steps:
step 1, storing processed data by the multi-micro-organ culture database, and calling related data from the multi-micro-organ culture database after storage;
step 2, researching and establishing a multi-agent reinforcement learning algorithm, integrating and analyzing micro-organ multi-modal data, extracting interaction system state and feature information, and converting the interaction system state and the feature information into functional quantitative indexes for evaluating an application, wherein the application comprises micro-organ interaction research and medicine screening; and
step 3: displaying functional quantitative index data after conversion by the multi-micro-organ visualization terminal, carrying out autonomous learning by the multi-micro-organ culture and interaction big data analysis system according to the related data after display, and then carrying out autonomous optimization, to complete multi-micro-organ culture and interaction big data analysis.
